(19) United States
(12) Patent Application Publication
(10) Pub. No.: US 2005/0214769 A1
(43) Pub. Date: Sep. 29, 2005

(54) Method for producing probe carrier, apparatus for producing the same and method for quality assurance therefor

(76) Inventors: Hiroyuki Hashimoto, Kanagawa (JP); Tadashi Okamoto, Kanagawa (JP); Mie Ishii, Tokyo (JP)

Correspondence Address: MORGAN & FINNEGAN, L.L.P., 3 WORLD FINANCIAL CENTER, NEW YORK, NY, 10281-2101, US

(21) Appl. No.: 10/519,813

(22) PCT Filed: Jun. 27, 2003
(86) PCT No.: PCT/JP03/08198

§ 371 Date: Dec. 27, 2004

(30) Foreign Application Priority Data
Jun. 28, 2002 (JP) 2002-191421

(51) Int. Cl. C12Q 1/68; G06F 19/00; G01N 33/48; G01N 33/50

(52) U.S. Cl. 435/6; 702/20

(57) ABSTRACT

To provide a technology for obtaining a probe carrier which is quality assured by an effective inspection program and an effective producing steps. Quality of a produced probe carrier is ensured by expanding test items in the processes from synthesis and purification of the probe to fixation thereof to a carrier.

### TECHNICAL FIELD

The present invention relates to a method for producing a probe carrier (also called a probe chip or a biochip) in which plural probes are arranged in a matrix shape on a carrier, a producing apparatus therefor, a quality assurance method therefor and a probe carrier produced and quality assured by such producing method and quality assurance method.

### BACKGROUND ART

Probe chips such as a DNA chip or a protein chip is becoming to be utilized in acquiring gene information such as genome analysis or analysis of gene expression, and the result of such analysis is expected to provide important indexes for diagnosis, prognosis prediction, determination of therapeutic principle etc. for cancer, hereditary diseases, life habit diseases, infections etc.

Certain methods are known for producing the aforementioned probe chip. As an example, in case of producing DNA chip, representative methods are:

(1) a method of synthesizing a DNA probe in successive manner by photolithography on a substrate (U.S. Pat. No. 5,405,783 (patent reference 1)); and

(2) a method of supplying and fixing DNA or cDNA (complementary DNA) synthesized in advance on a substrate (U.S. Pat. No. 5,601,980 (patent reference 2), Japanese Patent Application Laid-open No. 11-187900 (patent reference 3), Science Vol. 270, 467, 1995 (non-patent reference 1) and Nature Biotechnology Vol. 18, 438, 2000 (non-patent reference 2)).

The probe chip is generally produced in such methods, but, in case of using such probe chip in the aforementioned applications, in order to ensure the reliability, or quantitativeness and reproducibility of the analysis, it is important to know the amount or density of the probe present in probe fixation areas (also called spots or dots) mutually separated and constituting a matrix. It is also important, depending on the producing method for the chip, to know the actual shape (imaging) of the matrix (shape, size, and state). Also in case of supplying the probe chips in a large number, it is extremely important to know a fluctuation in the amount of the probes present in the aforementioned matrix within a production lot or between the productions lots and the purity of such probe.

The aforementioned producing method (1) is satisfactory for positioning probes of plural kinds on a chip, but,. since the probe is synthesized in succession on the substrate, it is impossible in principle to avoid a situation where probes shorter for example by a base in succession than a predetermined probe chain length and undesirable for the intended purpose are more or less present mixedly (as defects), also including in principle a case where the positions of such defects cannot be identified. Such problem, if once generated, leads to a lack of reliability of the probe chips produced by such method, since the probe formed on the substrate cannot in principle be purified. In such case, even if an aforementioned analysis is possible for the probe on thus produced chip, the significance thereof becomes low as the purification is not possible.

On the other hand, in the producing method (2), it is anticipated possible to improve the reliability of the chip, since there can be employed a probe which is obtained through a synthesis, a purification, and an inspection for concentration, purity etc.

However, the probe on the probe chip is present in principle in a level of a single molecular layer, and the size of a matrix dot has recently become as small as about 10 μm (in certain producing method), so that the probe is present in an extremely small amount in each matrix dot. For this reason, there is required a surface analysis technology of an extremely high sensitivity, for analyzing each matrix dot on the probe chip.

There are already known certain surface analysis technologies of such high sensitivity, but, for example a method of labeling the probe with an isotope is often not commonly acceptable since it is complex, involves a danger and requires special facilities and apparatus.

Another example is a method of applying fluorescent labeling to the probe, or a method of applying fluorescent labeling to a substance which causes a specific coupling with the probe, namely a fluorescent hybridization method utilizing a nucleic acid chip, is associated with problems such as a stability of the fluorescent dye, a quenching, a non-specific adsorption of the fluorescent dye to the substrate surface and a quantitative nature (stability and reproducibility) of a specific coupling (hybridization), and there may result a problem in quantitatively determining the amount of the probe itself.

(Patent reference 1)

U.S. Pat. No. 5,405,783

(Patent reference 2)

U.S. Pat. No. 5,601,980

(Patent reference 3)

Japanese Patent Application Laid-open No. 11-187900

(Non-Patent reference 1)

Science, Vol.270, 467, 1995

(Non-Patent reference 2)

Nature Biotechnology, Vol.18, 438, 2000

### DISCLOSURE OF THE INVENTION

The present inventors have made intensive investigations on the problems in such prior probe chip producing methods, producing steps, producing apparatuses or producing system and on the problems in quality assurance of the probe chip produced by such prior producing methods, producing steps, producing apparatuses or producing system, and have thus made the present invention.

An object of the present invention is to provide a technology for providing a probe carrier which is quality assured by an effective inspection program and an effective producing steps.

### BRIEF DESCRIPTION OF THE DRAWING

FIG. 1 is a drawing showing an example of a method of bonding a DNA probe to a glass substrate.

### BEST MODE FOR CARRYING OUT THE INVENTION

In a first embodiment of a producing method for a probe carrier of the invention, there is provided a method for producing a probe carrier in which plural probes are fixed on a surface of a carrier, the method including:

(8-1) a step of executing an analytical inspection on the surface of the carrier for judging “good” or “not good” state of the carrier according to the result of the analytical inspection and a predetermined criterion;

(8-2) a step of depositing at least one selected from plural probe solutions onto the carrier judged as “good” so as to form a probe deposition area independent for each probe solution;

(8-3) a step of executing an inspection concerning a formed state of the probe deposition area on the carrier on which the probe deposition area is formed, and judging “good” or “not good” state of the deposition according the result of the inspection and a predetermined criterion;

(8-4) a step of executing, on the carrier having the probe deposition area judged as “good”, a fixation of the probe to the surface of the carrier thereby obtaining a probe carrier;

(8-5) a step of executing an analytical inspection on the probe in at least one of the plural probe fixation areas constituted of probes fixed on the carrier; and

(8-6) a step of judging “good” or “not good” state of the produced probe carrier according to the result of the analytical inspection and a predetermined criterion.

More specifically, there is provided a method for producing a probe carrier, including:

(1) a step of preparing a purified probe;

(2) a step of obtaining probe information on the purified probe;

(3) a step of judging “good” or “not good” quality of each purified probe according to the obtained probe information and a predetermined criterion;

(4) a step of obtaining a probe of which quality is “good” in case of the purified probe of which quality is judged as “not good”;

(5) a step of individually dissolving each purified probe judged as “good” in a solvent for ejection to a carrier, based on at least a part of the probe information obtained in (2), in a predetermined concentration and storing each obtained probe solution in an individual storing container;

(6) a step of transferring each probe solution stored in the storing container to another container equipped in an apparatus for deposition onto the carrier;

(7) a step of applying a surface treatment for fixing the probe to the carrier;

(8) a step of depositing the probe solution onto a treated surface of the carrier by a method including following steps, thereby forming a plurality of mutually independent probe fixation areas;

(8-1) a step of executing an analytical inspection on the carrier for judging “good” or “not good” state of the carrier according to the result of the analytical inspection and a predetermined criterion;

(8-2) a step of depositing at least one selected from plural probe solutions onto the carrier judged as “good” so as to form a probe deposition area independent for each probe solution;

(8-3) a step of executing an inspection concerning a formed state of the probe deposition area on the carrier on which the probe deposition area is formed, and judging “good” or “not good” state of the deposition according the result of the inspection and a predetermined criterion;

(8-4) a step of executing, on the carrier having the probe deposition area judged as “good”, a fixation of the probe to the surface of the carrier thereby obtaining a probe carrier;

(8-5) a step of executing an analytical inspection on the probe in at least one of the plural probe fixation areas constituted of probes fixed on the carrier; and

(8-6) a step of judging “good” or “not good” state of the produced probe carrier according to the result of the analytical inspection and a predetermined criterion.

In a second embodiment of the invention, there is provided a method for producing a probe carrier including:

(a) a step of designing plural kinds of probes for detecting a target substance;

(b) a step of synthesizing the designed plural probes;

(c) a step of individually purifying the synthesized plural probes;

(d) a step of obtaining probe information on each purified probe;

(e) a step of judging “good” or “not good” state of synthesis and purification in each purified probe according to the obtained probe information and a predetermined criterion;

(f) a step of repeating the foregoing steps (b) to (e) on the purified probe of which the state of synthesis and purification is judged as “not good”, thereby obtaining “good” state of synthesis and purification in all the purified probes;

(g) a step of individually dissolving each purified probe judged as “good” in a solvent for ejection to a carrier, based on at least a part of the probe information obtained in (d), in a predetermined concentration and storing each obtained probe solution in an individual storing container;

(h) a step of transferring each probe solution stored in the storing container to another container equipped in an apparatus for deposition onto the carrier;

(i) a step of applying a surface treatment for fixing the probe to the carrier;

(j) a step of depositing the probe solution onto a treated surface of the carrier by a method including following steps, thereby forming a plurality of mutually independent probe fixation areas;

(j-1) a step of executing an analytical inspection on the carrier for judging “good” or “not good” state of the carrier according to the result of the analytical inspection and a predetermined criterion;

(j-2) a step of depositing at least one selected from plural probe solutions onto the carrier judged as “good” so as to form a probe deposition area independent for each probe solution;

(j-3) a step of executing an inspection concerning a formed state of the probe deposition area on the carrier on which the probe deposition area is formed, and judging “good” or “not good” state of the deposition according the result of the inspection and a predetermined criterion;

(j-4) a step of executing, on the carrier having the probe deposition area judged as “good”, a fixation of the probe to the surface of the carrier thereby obtaining a probe carrier;

(j-5) a step of executing an analytical inspection on the probe in at least one of the plural probe fixation areas constituted of probes fixed on the carrier; and

(j-6) a step of judging “good” or “not good” state of the produced probe carrier according to the result of the analytical inspection and a predetermined criterion.

Also according to a first embodiment of a producing system for a probe carrier of the present invention, there is provided a producing system to be employed in the aforementioned producing method for the probe carrier, the system including:

an analyzing apparatus for obtaining probe information of each purified probe;

an inspection apparatus for judging “good” or “not good” state of synthesis and purification in each purified probe;

an apparatus for depositing the probe solutions onto the carrier from the storing containers respectively and individually storing the purified probe solutions judged as “good”;

an analyzing apparatus for the carrier subjected to the aforementioned surface treatment;

an inspection apparatus for judging “good” or “not good” concerning a formed state of the probe deposition area on the carrier on which the probe deposition area is formed;

an apparatus for executing, on the carrier having the probe deposition area judged as “good”, a fixation of the probe to the surface of the carrier thereby obtaining a probe carrier; and

an apparatus for executing an analytical inspection on the probe in at least one of the plural probe fixation areas constituted of probes fixed on the carrier.

In a second embodiment of a producing system for a probe carrier of the present invention, there is provided a producing system to be employed in the aforementioned producing method for the probe carrier, the system including:

a synthesizing apparatus for synthesizing designed plural probes;

a purifying apparatus for individually purifying the synthesized plural probes;

an analyzing apparatus for obtaining probe information on each purified probe;

an inspection apparatus for judging “good” or “not good” state of synthesis and purification in each purified probe;

an apparatus for depositing the probe solutions onto the carrier from the storing containers respectively and individually storing the purified probe solutions judged as “good”;

an analyzing apparatus for the carrier subjected to the aforementioned surface treatment;

an inspection apparatus for judging “good” or “not good” concerning a formed state of the probe deposition area on the carrier on which the probe deposition area is formed;

an apparatus for executing, on the carrier having the probe deposition area judged as “good”, a fixation of the probe to the surface of the carrier thereby obtaining a probe carrier; and

an apparatus for executing an analytical inspection on the probe in at least one of the plural probe fixation areas constituted of probes fixed on the carrier.

A quality assurance method for a probe carrier of the present invention is featured in executing an analytical inspection, utilizing the aforementioned producing method or the aforementioned producing system, on any or all of a probe in the probe solution prior to the deposition onto the carrier, a surface treated carrier, a probe deposition area after the deposition of the probe solution, and a probe fixed to the carrier after the deposition of the probe solution, thereby assuring the quality of the probe chip. Data of such analytical inspection are preferably present in respect of at least one of the plural probe fixation areas on the probe carrier.

The probe carrier of the present invention includes a probe carrier produced by the aforementioned producing method or the aforementioned producing system, and a probe carrier of which a quality is assured by the aforementioned quality assurance method.

In the present invention, in the first embodiment of the producing method and the first embodiment of the producing system for the probe carrier, a prepared and purified probe is subjected to a quality inspection according to a predetermined criterion. In case the probe information obtained from the purified probe does not depend on a degree of purification, the step for obtaining the probe information can be executed in a state in which the degree of purification is not judged. In case it depends on a degree of purification, there can be employed a method of inspecting the quality according to a criterion concerning the degree of purification, and obtaining the probe information when a probe of a predetermined degree of purification becomes available.

The probe carrier of the present invention is featured in that it is associated with analytical data on at least one of the probe fixation areas or quality assurance data on the entire carrier, and such data may be provided in various forms such as a print on paper or electronic file data in a medium, and may be provided separately from the probe carrier or integrally therewith.

Also in case the probe fixation areas are arranged in a matrix shape, the aforementioned data for plural probes may be attached as image data.

According to the present invention, there can be provided a technology for obtaining a probe carrier which is quality assured by an effective inspection program and effective producing steps.

(Embodiments of the Invention)

In the present invention, a probe fixed on the carrier is capable of specific coupling with a specified target substance. For example, for a nucleic acid such as DNA or RNA, a probe having a base sequence complementary to that of a target nucleic acid can form a hybrid therewith.

In the present invention, a probe carrier means a carrier having thereon a probe fixation area for example of a dot shape in which a nucleic acid probe is fixed in a dot or spot shape, and a probe array means a carrier on which plural or multiple probe fixation areas are mutually independently arranged in predetermined positions on the carrier, for example in a matrix shape. Such probe carrier generally includes nucleic acid chips such as a microarray, a probe chip, a DNA chip or an RNA chip.

The carrier can be selected from various materials and various shapes, and, for example, a glass substrate, a silicon substrate or a metal substrate can be advantageously employed.

In the following, based on the nature of the present invention and on certain specific embodiments and examples of the present invention, there will be explained means of solving the problems in the prior art and embodiments of the present invention.

The present invention includes a producing method for a probe carrier (hereinafter called probe chip) basically constituted of all or a part of following steps; another producing method utilizing the aforementioned producing method as a part of steps; a producing apparatus (apparatuses) or a producing system for such producing methods; a quality assurance method for a probe chip produced by such producing methods or by such producing apparatus (apparatuses) or producing system; and a probe chip produced and quality assured by such producing methods, such producing apparatus (apparatuses) or producing system.

More specifically, a preferred embodiment of the producing method for the probe chip of the present invention include following steps:

(1) a step of designing plural kinds of probes;

(2) a step of synthesizing the designed plural probes;

(3) a step of purifying the synthesized plural probes;

(4) a step of obtaining probe information on the synthesized and purified plural probe;

(5) a step of judging “good” or “not good” state of “synthesis and purification” in each of the plural probes, according to the obtained probe information and a predetermined criterion;

(6) a step of repeating the foregoing steps (2) to (5) on a single probe or plural probes of which the state of “synthesis and purification” is judged as “not good”, thereby obtaining “good” state of “synthesis and purification” in all the probes;

(7) a step of individually dissolving the plural probes of which the “synthesis and purification” state is judged as “good” in solvents for spotting, based on at least a part of the probe information obtained in (4), at a predetermined concentration and storing probe solutions in respective storing containers;

(8) a step of transferring parts (in quantity) of the plural probe solutions stored in the containers to other containers equipped in an apparatus for spotting;

(9) a step of applying a surface treatment for fixing the probe to a substrate for spotting;

(10) a step of executing an analytical inspection on the substrate subjected to the surface treatment and judging “good” or “not good” state of the substrate according to a result of the analytical inspection and a predetermined criterion;

(11) a step of spotting all (in kinds) or a part (in kinds) of the plural probe solutions judged as “good” in a matrix shape on the substrate judged as “good”;

(12) a step of executing an inspection concerning the spotting on the substrate subjected to the spotting, and judging “good” or “not good” state of the spotting according to a result of the inspection and a predetermined criterion;

(13) a step of applying, to the substrate subjected to the spotting and judged as “good”, a treatment necessary for fixation of the probe to the substrate;

(14) a step of executing an analytical inspection on the probes in a part or all of the spots, fixed to the substrate; and

(15) a step of judging “good” or “not good” state of the produced probe chip according to a result of the analytical inspection and a predetermined criterion.

In the following there will be given a detailed explanation on each of the probe chip producing steps, on the apparatus (apparatuses) to be used therein, on the probe chip obtained through producing steps utilizing such apparatus (apparatuses) and on the quality assurance method for such probe chip.

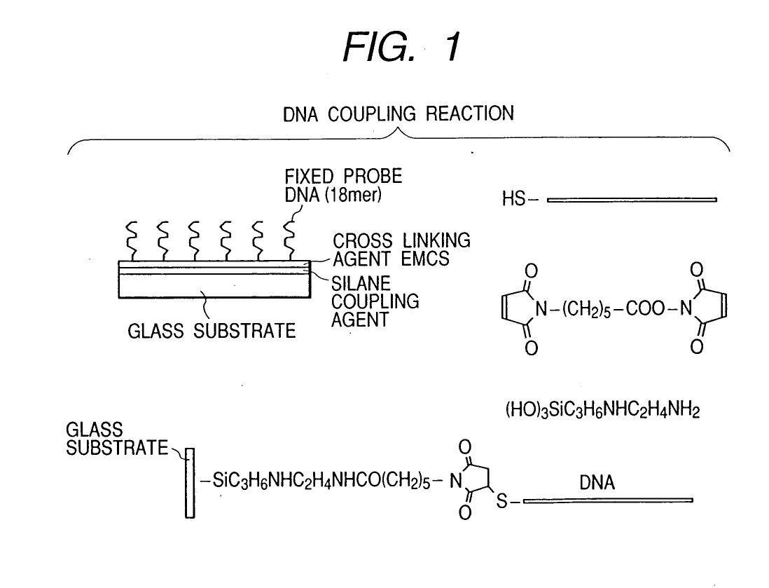

FIG. 1 is a view schematically showing an example of a method for coupling the probe to the substrate, in the probe chip producing steps of the present invention. In the drawing, a probe is represented by an 18mer oligonucleotide (DNA: single-strand nucleic acid), but the probe in the present invention is not limited to an oligonucletide and can also be constituted by another nucleic acid (such as cDNA), a nucleic acid analog such as peptide nucleic acid (PNA), an oligopeptide or a protein. An important factor in the present invention is that, for such substance, a designing; a synthesis according to the designing, a purification and an certification for purity can be executed in advance prior to the coupling with the substrate.

Designing of a probe means selecting, as a probe, a base sequence which is basically complementary, but in certain cases not complementary in certain bases, to a nucleic acid base sequence of a substance which specifically combines with thus designed probe, or, in case of a nucleic acid probe, a portion to be investigated such as a genome DNA, a gene DNA or an mRNA, or selecting an amino acid sequence in case of a peptide or a protein. Also a selection of a length of the probe is a part of such probe designing, and selection of a linker or a functional group required for a covalent bonding in fixing the probe to the substrate is included in the probe designing in a wider sense.

FIG. 1 shows an example of a method for covalent bonding a synthesized oligonucleotide on a glass substrate. In this method, the glass substrate is treated with a silane coupling agent having a primary amino group (KBM-603; manufactured by Shinetsu Chemical Industries; illustrated structure being after hydrolysis), and then a succinimide ester group of a bifunctional crosslinking agent N-(4-maleimidobutyryloxy)succinimide (EMCS: manufactured by Dojin Chemical Laboratories) is reacted with an amino group of the aforementioned silane coupling agent. Finally, an oligonucleotide probe having an appropriate base sequence and provided at an end with a thiol (SH) group across a linker (linker structure being omitted in the illustration) is bonded by a reaction between the thiol group thereof and a maleimide group of EMCS, whereby the oligonucleotide is covalent bonded to the glass substrate. In the illustration, the bonding between the silane coupling agent and the glass substrate, in a portion where the end of the probe is eventually bonded to the glass substrate, is illustrated only in a schematic manner.

In the present invention, there is produced a probe chip having, on a substrate, plural kinds of probes are arranged in a matrix shape. For depositing (spotting) plural kinds of probe solutions on the substrate, there can be employed, for example, a pin method, a microsyringe method, or an ink jet method such as a piezo ink jet method or a thermal ink jet method.

As already explained in the foregoing, one of the features of the present invention is to employ a probe subjected in advance to synthesis, purification and certification for example of purity, also to adequately monitor each process of the chip production, to execute an analytical inspection on the produced chip and to mutually correlate the results of such certification, analysis and inspection thereby managing the steps of the production, and executing a feedback in certain cases thereby finally assuring the quality of the chip.

In the foregoing process, a probe of a relatively large amount can be simply and precisely purified by an HPLC method (high performance liquid chromatography), depending on an apparatus and a column to be used. The HPLC (high performance liquid chromatography) is one of the means for separating a mixture containing organic compounds including biomaterials, and is a chromatography utilizing a liquid as the moving phase.

One of the features of the present invention is to obtain information from thus purified probe thereby judging “good” or “not good” state of the probe and determining whether or not to proceed to a next step, and the probe information to be obtained is firstly an yield, namely a weight of the synthesized and purified probe. As the amount of the probe after synthesis is usually very small, it is practical, rather than measuring the actual mass, to measure a concentration of the probe dissolved and diluted at a predetermined proportion, and, for this purpose, there can be employed an ordinarily utilized method such as an optical absorption method. Since the nucleic acid has an absorption maximum at a wavelength around 260 nm, which is slightly variable depending on the base sequence, it is possible to determine the concentration of the probe solution or the yield thereof, by measuring the optical absorbance at such wavelength and comparing it with a molar optical absorption coefficient, calculated in advance, of the probe.

Second probe information is a purity of the probe. In case of purifying the probe, for example an oligonucleotide, with the HPLC, there may be contained as the impurities at the time of synthesis oligonucleotides shorter by one base, and if they have eluting times of HPLC close to that of the probe of the desired chain length, the content of the impurities after the purification may become relatively high, depending on such closeness of the eluting time and on a fraction collecting range at the purification. In order to prepare for such situation, it is necessary to quantitatively confirm the purity of the probe after the purification. Such confirmation can be achieved by the HPLC method, or an LC-MS method (liquid chromatography-mass spectrometry) employing a mass spectrometer as a detector for liquid chromatography, or a MALDI method (matrix assisted laser desorption ionization time-of-flight mass spectrometry).

For the mass spectrometer constituting the detector for the LC-MS method, there can be employed a quadrupole type, an ion trap type, a time-of-flight type, a magnetic field type, a Fourier transformation ion cyclotron resonance type or an FT-ICR type. Also an ionizing method employed in the LC-MS can be an electrospray ionization (ESI) method or an atmosopheric pressure chemical ionization (APCI) method. In the present invention, the analysis is achieved by suitably utilizing these methods.

In the following, there will be briefly explained the principle of measurement by the MALDI method.

At first, a specimen and an easily ionizable substance, called matrix, are mixed and placed on a specimen table, and are irradiated with a pulsed laser light of wavelength absorbable by the matrix, thereby causing ionization of the matrix. Almost simultaneously, the specimen to be analyzed is ionized by the transfer of energy from the matrix. (Such energy transfer proceeds instantaneously. The mechanism of ionization of the specimen in the co-presence of the matrix is not exactly known.) A strong electric field is applied onto the specimen table, whereby the ionized specimen is guided, together with the ionized matrix, to a time-of-flight mass spectrometer. As a lighter ion flies faster while a heavier ion flies slower, a mass of the generated ion can be analyzed by measuring a time from the ion generation to the detection (time of flight). The ionization method employed in the MALDI is known as a soft ionization method which is less likely to cause a damage or a cleavage in the specimen to be analyzed. Also certain commercial MALDI apparatus is capable of measuring plural specimens in continuous manner.

The MALDI method, ionizing the specimen under a relatively mild condition, can analyze the mass of an ion itself of the specimen. More specifically, a probe analysis by the MALDI method, for example in case of an oligonucleotide probe, provides a different of 300 mu (mass unit) or larger for a difference by a base, so that separation and analysis can be achieved easily and securely even in a quantitative manner.

In the present invention, these methods are used for certifying the purity of the probe, thereby judging whether or not to use the synthesized and purified probe in the chip production, but the criterion for purity judgment varies depending on the purpose of use of the probe chip. More specifically, in case of merely investigating the presence or absence of a tested material, a purity of about 80% may be acceptable, but, in case a strict quantitativeness is required, there is required a purity of 90%, preferably 95% or higher.

Third probe information is naturally information as to whether a desired probe is obtained, that is, in case of a nucleic acid probe, whether a base sequence, a chain length and other structures are obtained, and in case of a protein probe, whether an amino acid sequence, a chain length and other structures are obtained. In strict sense, such information should be obtained by exact analysis of the sequence, but, since such operation is extremely cumbersome, a mass analysis may be employed in the present invention as alternative means. For such mass analysis, the aforementioned MALDI method can be employed. However, such mass analysis can only provide a composition of bases in case of a nucleic acid or a composition of amino acids in case of a protein, instead of the base sequence or the amino acid sequence itself. Nevertheless, such information is useful since a mass if different from a predetermined value indicates that a desired probe is not synthesized.

The MALDI method is known to show certain fluctuation in the data of analysis depending on the preparation of specimen and the level of mass calibration. For example, in case of a nucleic acid, the bases have certain differences in the mass number, such as 135.1 for adenine, 151.1 for guanine, 111.1 for cytosine, and 126.1 for thymine. Therefore, in the present invention, that a criterion for judging “good” or “not good” is suitably selected, such as a measured mass number being within ±2.0 amu (atomic mass unit), ±1.0 amu, ±0.5 amu or ±0.1 amu of a theoretical mass number of the probe, and the quality of the probe is judged based on the measured mass number of the probe and the theoretical mass number of the probe calculated in advance. For the mass analysis of the probe, the aforementioned MALDI method is advantageous, but a TOF-SIMS method (time-of-flight secondary ion mass spectrometry), which will be explained later in more details, is recently showing a remarkable technical progress and is becoming highly sensitive and capable of analyzing a high mass number by employing gold ion or polyvalent gold ion as a primary ion, and an analysis of a total mass number of the probe is also possible with such method.

The total mass number of a probe is analyzed by the aforementioned methods and is compared with a theoretical mass number, and, in case of a nucleic acid, the theoretical mass number is a sum of mass numbers of the nucleotides. In case a linker etc. is coupled to the nucleic acid probe, the mass number naturally shows a corresponding change, but a difference in the mass number between the probes basically results from a difference in the mass numbers between the nucleotides, namely from an aforementioned difference in the mass number between the bases. In case the probe is constituted of a potein or an oligopeptide, the difference in the mass number of the probe results from a different in the mass number of amino acids. In the following, the mass analysis will be explained, taking a case of a nucleic acid for the purpose of simplicity, but, in case of employing the above-explained method, an identical total mass number may include different compositions of nucleotides, depending on the precision of mass analysis (mass resolution) of the total mass number of the analyzed nucleic acid probe and the precision of calculation of the mass number of the nucleotide, calculated in advance.

TABLE 1

| | Nucleotide chain length | | | | |
|---|---|---|---|---|---|
| | 5 | 10 | 20 | 30 | 40 |
| number of cases | 56 | 286 | 1771 | 5456 | 12341 |
| precision (first digit in | *56 | 251 | 985 | 2838 | — |
| fraction) | | | | | |
| precision (second digit | 56 | *286 | *1771 | *5456 | 12301 |
| in fraction) | | | | | |
| precision (third digit in | 56 | 286 | 1771 | 5456 | *12341 |
| fraction) | | | | | |
| precision (fourth digit | 56 | 286 | 1771 | 5456 | 12341 |
| in fraction) | | | | | |

Table 1 shows, for each of a nucleotide chain length of 5-, 10-, 20-, 30- and 40-mer, a number of possible cases of the nucleotide composition and a precision of the mass number of the nucleotide required to distinguish all the aforementioned cases. Table 1 indicates that, for a nucleotide chain length of 5-, 10-, 20-, 30- or 40-mer, all the nucleotide compositions can be distinguished by determining the mass number of each nucleotide with a precision respectively to the first, second, second, second or third place in the fraction. In the present invention, in order to distinguish the probes which may be calculated as having a same mass number, the analysis, calculation and identification are executed with an improved precision in the mass analysis and the precision of calculation of the mass number of the nucleotide. For example, for 20mer oligonucleotides, all the probes can be distinguished and identified by executing the mass analysis and the calculation of the mass of the nucleotide with a precision to the second place of the fraction. The chain length of the probe is determined according to the purpose of use, and the necessary precision of the mass analysis and the mass calculation of the nucleotide is determined accordingly.

The precisions of the mass analysis and the mass calculation are preferably equal to or higher than the precisions required for specifying the composition of nucleotides or amino acids in the probe. Such precisions, namely a precision of mass resolution in the total mass number of the probe and a precision of calculation of the mass number of nucleotides or amino acids, are preferably a first place in the fraction or higher, more preferably a second place in the fraction or higher, further preferably a third place in the fraction or higher, and most preferably a fourth place in the fraction or higher.

In case the results of mass analysis cannot be obtained with such precision, it is possible to identify the nucleotide composition of the probe even without the aforementioned precisions of the mass analysis and the mass calculation of the nucleotides, by providing another information such as a CG content (a summed content of cytidylic acid and guanylic acid) of the nucleotide of the probe, and the present invention executes such process when necessary.

The above-described method, for identifying the probe by specifying the nucleotide composition or the amino acid composition of the probe from the analysis of total mass of the probe and from the theoretical mass number of the nucleotides or amino acids constituting the probe, can be employed not only for judging the good or not good state of the probe but also in the identification of a probe after spotting or of a probe fixed on the substrate, as will be explained later.

The synthesis of the probe and the confirmation of the synthesis are executed through the above-described steps. In the present invention, in order to ensure the probe finally fixed on the chip, the above-described steps are repeated again in this stage for any probe that is judged as “not good” in the synthesis, yield of purity, thereby eventually bringing all the probes to a “good” state.

Then, an actual chip is prepared by the probe judged as “good” in the synthesis, yield and purity. In this operation, the probe judged as “good” is dissolved with a suitable concentration and is spotted on a substrate subjected to the aforementioned surface treatment. The concentration in this operation is determined for example by a spotting apparatus, a drop amount of a spotting liquid, a desired spot diameter; a density, a reactivity and a reaction time of a functional group on the substrate surface to be coupled with the probe, and an yield of the probe. An excessively high concentration is unnecessary in consideration of the economy and the quality of the produced chip, while an excessively low concentration is undesirable as it lowers the density of the probes formed on the chip. Based on the foregoing, the probe concentration in the present invention is suitably selected within a range not exceeding 200 μM. More specifically, it may be selected as 100 μM or less, 50 μM or less, 20 μM or less, 10 μM or less, or 5 μM or less. In certain cases, there may be selected a range of 1 μM or less.

Probes of plural kinds, dissolved in a suitable concentration and a suitable amount, are stored in appropriate containers in preparation for a spotting in a next step, but, in the present invention, such probe solutions may be stored in a frozen state in such containers, and transferred from such container to a spotting apparatus by a dispensing apparatus. As the chip of the present invention bears probes of 100 to 1000 kinds or even larger according to the requirement, the container for storing the probes is preferably a microplate having 96, 128, 384 or 1536 recesses (wells).

In the following, there will be given a detailed explanation on a substrate to be employed in the chip of the present invention, and a processing for fixing the probe to the substrate.

An example of the fixing method of the probe onto the chip to be employed in the present invention is already shown in FIG. 1, but, merely from the standpoint of probe fixation, the substrate for fixing the probe may be of any material or any shape, and such substrate may also be employed in the present invention in certain cases. On the other hand, one of the features of the present invention is to analyze the substrate prior to the probe fixation or the substrate after the probe fixation, thereby assuring the quality of the chip, it is preferred that the material, the shape and the surface state of the-substrate are suitable for such analysis. In this sense, there is preferred a rigid substrate that can be worked to obtain a smooth surface, such as a glass substrate, a silicon substrate or a metal substrate. Also a property of the substrate such as a conductivity may affect the result of analysis depending on the method of chip analysis as will be explained later, it is desirable to suitably select a glass substrate, a silicon substrate or a metal substrate in such case, according to the method of analysis. More specifically, the glass substrate can be suitably selected among different kinds such as quartz and Pyrex, as they show different properties.

The fixation of the probe to the substrate may be achieved by any method as long as the probe or the fixing method can be analyzed, and there may be employed a non-covalent bonding such as an adsorption or an electrostatic coupling of the probe to the substrate surface, but a covalent bonding as shown in FIG. 1 may be preferred because of stability and reproducibility of the analysis, in case the analysis involves an ionization of the probe as will be explained later. It is also desired that the probe is fixed by a covalent bonding to the substrate, in certain modes of use of the chip, for example in case the chip is placed in a high temperature state.

In the present invention, an analysis is executed for judging “good” or “not good” state of a surface treatment applied to the substrate for the purpose of chip production, and such surface treatment means a washing of the substrate, or a treatment required for fixing the probe, such as a treatment required for a covalent bonding in case the probe is fixed by a covalent bonding. After such treatment, a suitable analysis is executed to judge “good” or “not good” state of such surface treatment.

A first method that can be conceived for such surface analysis is a measurement of a contact angle. The contact angle measurement, though not explained in detail, is to represent a surface state such as a wetting property by an angle formed between such surface and a liquid drop placed on such surface. Ideally, a surface subjected to a specified surface treatment shows a specified contact angle with respect to a specified liquid (for example water). The measurement of the contact angle is executed at a predetermined time selected after the washing step and steps required for probe fixation.

For example, in the present invention, a “good” or “not good” washed state of the substrate surface is judged by a contact angle. Criterion for judgment is selected according to a material constituting the substrate, a washing method and a required washed state, and, in the present invention, it is selected at 10° or less, 8° or less or 6° or less, in stepwise manner according to the case. A contact angle of 5° or less is difficult to set as a criterion since the liquid spreads and the measurement becomes not reproducible and not reliable. As to a surface treatment other than washing, such surface treatment increases the contact angle in comparison with a washed substrate, a range of about ±2.5° with respect to such specific contact angle is taken as a criteria for judging “good” or “not good” of the surface treatment.

As other methods for analyzing the surface treated substrate in the present invention, there can be employed a TOF-SIMS method or an XPS method (X-ray photoelectron spectroscopy).

The TOF-SIMS method, known as a method of mass analysis, is to investigate atoms or molecules present on the outermost surface of a solid sample, and has following features, such as a detecting ability of a trace amount component of 10⁹ atom/cm² (corresponding to 1/10⁵ of an outermost one-atom layer), adaptability to both of an organic substance and an inorganic substance, ability to measure all elements and compounds present on the surface, and ability of an imaging of secondary ions from a substance present on the sample surface.

In the following, the principle of this method will be explained briefly. When a solid sample surface is irradiated with a high-speed ion beam (primary ions) in a high vacuum, a component constituting the surface is emitted to the vacuum by a sputtering phenomenon. Positively or negatively charged ions generated in this state (secondary ions) are concentrated in a direction by an electric field, and are detected at a position separated by a predetermined distance. In the sputtering, there are generated secondary ions of different masses according to the composition of the sample surface, and, since lighter ions fly faster while heavier ions fly slower, the mass of the generated secondary ion can be analyzed by measuring a time of flight from the generation of the secondary ions to the detection.

While a dynamic SIMS method which is known as a similar method can provide a limited information on the chemical structure from a mass spectrum since an organic compound is decomposed at an ionization to fragment ions or to particles, the TOF-SIMS method, employing a very low amount of primary ions irradiated, allows to ionize an organic compound in a state of relatively maintaining the chemical structure whereby the structure of the organic compound can be known from the mass spectrum. Also since only the secondary ions generated in the outermost surface of the solid sample are emitted into the vacuum, it is possible to obtain information of the outermost surface (a depth of several Angstroms) of the sample.

The TOF-SIMS apparatus is largely divided into a sector type and a reflectron type, and one of the differences between these types is an electrical method of grounding a holder for fixing the sample to be analyzed. In the sector type, because of the configuration of the apparatus, a positive or negative voltage of several kilovolts is applied to such holder thereby guiding the secondary ions to a mass spectrometer, while, in the reflectron type, the holder is grounded and a positive or negative voltage of several to several tens of kilovolts is applied to a secondary ion extracting electrode to guide the secondary ions to the mass spectrometer.

The TOF-SIMS method usually utilizes positive primary ions, but positive secondary ions and negative secondary ions are generated regardless of the polarity of the primary ions. Also in an ordinary measuring condition, secondary electrons are generated by the irradiation with the primary ions regardless of the polarity thereof, and, since the generated amount of such secondary electrons is larger than the amount of the primary ions, the surface of the measured sample tends to be charged positively, whereby the measurement may be hindered in case such charge becomes excessive (so-called charge-up state). In consideration of the configuration of the apparatus, such positive charge may become largest in case of measuring negative secondary ions of an insulating substance by means of the sector type apparatus (since all the generated secondary electrons are directed toward the second ion extracting electrode under the aforementioned positive voltage).

In order to neutralize such positive charge, both the sector type and the reflectron type are often equipped with a pulsed electron gun for charge neutralization. The charge neutralization with such electron gun is achieved by an electron beam irradiation on the analyzed sample for a predetermined time by such electron gun, after the primary ion irradiation (by a pulse of sub nanosecond or several nanoseconds) and the measurement of the time-of-flight of the positive or negative secondary ions and before the next pulsed irradiation of the primary ions. During such electron beam irradiation on the analyzed sample, the sample holder in the sector type or the secondary ion extracting electrode in the reflectron type is disconnected from the voltage application and grounded.

This method mostly relaxes (or annuls) the aforementioned positive charge and enables an analysis of an insulating material, but, the positive charging becomes strongest in case of measuring the negative secondary ions on the insulating substance by means of the sector type apparatus as explained in the foregoing, so that the margin for the charge neutralization becomes narrowest in this case. In any case, in order to avoid the charge up phenomenon, it is generally more advantageous to use a reflectron type apparatus in which the sample holder is electrically grounded, than to use a sector type apparatus. Particularly in case the analyzed sample has a low conductivity (namely a higher resistivity or a higher dielectric constant), for example a glass, the reflectron type is suited better for the measurement.

On the other hand, the XPS method is to irradiate an analyzed sample with a soft X-ray in an ultrahigh vacuum and to analyze a kinetic energy of photoelectrons emitted from the surface by a photoelectric effect, and can determine a kind of an element and an oxidation state (chemical bonded state) thereof from a peak position of the photoelectrons as well as an approximate element composition (ratio) on the surface from an areal ratio of the peak areas. The XPS method can analyze a depth of several nanometers, and can measure a solid sample in non-destructive manner and can measure all the elements other than hydrogen, but a detection limit is about 0.1 atomic %. Although a simple comparison is difficult because of a difference in the depth of analysis, but it is inferior in sensitivity by 2 to 3 digits to the TOF-SIMS method. On the other hand, the TOF-SIMS method has a high sensitivity and can analyze all the elements including hydrogen, but, since this method is basically a destructive analysis, it has a drawback that the quantitative precision is not high.

The present invention analyzes the substrate surface after the surface treatment, suitably selecting these methods according to the situation and qualitatively or quantitatively determining a washed state, presence of an impurity, presence of a surface treated layer, a bonded state of a surface treated layer etc. and judging “good” or “not good” state of the surface treatment.

For example, in case of applying the XPS method for the above-explained purpose in the substrate shown in FIG. 1, there can be utilized at least one photoelectron peak area such as of Si 2p, C1s, 0.1s or N1s or an areal ratio of at least two as an index for such “good/not good” judgement. More specifically, nitrogen present in a silane coupling agent or in a bifunctional crosslinking agent is usually not contained in a glass substrate, an absolute intensity (peak area) of an N1s peak emitted from the substrate surface treated with a silane coupling agent or a bifunctional crosslinking agent, or a N1s/Si 2p ratio (peak area ratio) obtained by dividing such intensity with a Si 2p peak area provides an important index for “good/not good” judgment.

Also an element such as Na, S or Cl, which tends to stick to the substrate as a contamination in the course of a substrate treating process, can be detected with the XPS method with a relatively high sensitivity, so that high electron peaks of such elements (for example Na is, S 2p, Cl 2p etc.) can also be utilized for “good/not good” judgment.

The TOF-SIMS method, inferior in the quantitativeness as explained in the foregoing, has to be used carefully in the above-described purpose, but, in case of utilizing the TOF-SIMS method because of its high sensitivity, an intensity of secondary ions resulting from an impurity such as halogen ions or alkali metal ions can be used as an index for the “good/not good” judgment. Also an intensity or a relative intensity of secondary ions resulting from the silane coupling agent or the bifunctional crosslinking agent can be used as an index for the “good/not good” judgment. Furthermore, an intensity of secondary ions resulting from a carrier, such as glass, can also be used as an index for the “good/not good” judgment. Thus, at least one of such secondary ions can be used as an index for the “good/not good” judgment.

Also for the analysis of the surface treatment of the substrate, there can be employed an ellipsometry method. The XPS method and the TOF-SIMS method, requiring an ultra high vacuum for the measurement, are associated with drawbacks of requiring a relatively long time for the measurement and incapable of analyzing a large number of samples at a time, and the TOF-SIMS method is also associated with a drawback of being basically a destructive analysis. On the other hand, the ellipsometry method is capable of analysis in an ordinary environment and has an advantage of being a non-destructive analysis, so that it is suitably employed in the analysis in the present invention.

Then, in the present invention, the aforementioned probe judged as “good” is suitably formed into a solution and is spotted onto the substrate after the surface treatment, judge as “good”, whereupon the probe is fixed to the substrate, and, in such operation, the spotting means has to satisfy a “good” judging criterion regarding a state of a liquid drop after the spotting and a state of the probe finally bonded to the substrate.

In the present invention, the means for spotting the probe is selected suitably, according to the aforementioned judging criterion, from an apparatus equipped with single or plural pins, an apparatus equipped with single or plural microsyringes, and an apparatus equipped with single or plural ink jet nozzles. For such ink jet nozzle, there can be advantageously employed a piezo ink jet nozzle or a thermal ink jet nozzle.

In case the number of such plural pins, plural microsyringes, plural ink jet nozzles, plural piezo ink jet nozzle or plural thermal ink jet nozzles is less than the number of the kinds of the plural probes, there may be undesirably deteriorated the quality of a liquid drop after the spotting or a state of the fixed probe as will be explained later because of an influence by a re-filling of the probe solution or a time required therefor, so that the number of pins, microsyringes, ink jet nozzles, piezo ink jet nozzles or thermal ink jet nozzles is preferably equal to or larger than the number of kinds of the probes, for example 100 or larger, 500 or larger, 1000 or larger or 2000 or larger. A spotting apparatus may become difficult to prepare in the pin method or in the microsyringe method in case such number exceeds a certain level. On the other hand, a piezo ink jet nozzle or a thermal ink jet nozzle is suitable for preparing a multi-nozzle apparatus, as will be understandable from an ink jet printer, since the spotting apparatus can be prepared by a microworking technology.

The above-mentioned microworking technology allows, in certain cases to provide an assembly (head) of the ink jet nozzles, integrally with containers for the aforementioned probe solutions to be transferred, corresponding to the number of such ink jet nozzles. The probe to be employed has gone through a synthesis, a purification and an authentication for the purity, and its amount of use is desirably made necessary minimum in consideration of the cost, but, in the present invention, the capacity of the aforementioned container and the amount of the probe solution to be employed are determined suitably according to an yield of synthesis, a concentration to be used, a liquid amount to be spotted, a number of the chips to be produced, a configuration of the ink jet head etc. The capacity of the container can be suitably selected within a range from 100 μl or less to about 1 μl. For example, there can be preferably selected a capacity of 100 μl or less, 50 μl or less, 20 μl or less, 10 μl or less, 5 μl or less, 2 μl or less or 1 μl or less.

The above-described apparatus for probe spotting is preferably equipped with containers for containing the probe solutions, corresponding to the number of the ink jet nozzles, piezo jet nozzles or thermal jet nozzles.

After the spotting of the probe solution onto the substrate as explained in the foregoing, there is executed a next step of inspecting the spotted liquid drop.

A concentration of the probe in the probe solution is preferably 200 μM or less, and can be selected for example as 100 μM or less, 50 μM or less, 20 μM or less, 10 μM or less, or 5 μM.

Items of inspection of the spot are important as they directly affect the final quality of the chip, and can include, for example, presence/absence of a spot, a diameter of the spot, a shape of the spot, a spotted liquid amount, a spot position, presence of fine spot which should be absent, and presence/absence of associated dusts. It is also important whether a desired probe is present in a desired position.

In the present invention, a criterion for judging “good” or “not good” state is provided for each item, and the process proceeds to a next step if the result of “good”. A judging criterion is selected, for example, for the spot diameter, as within ±10% of a predetermined diameter; for the spot shape, as within a certain circularity; and for a spot arrangement, within ±10% of a spot diameter with respect to a predetermined line of arrangement. For inspecting an amount of a spotted liquid drop, there can be employed a method of three-dimensionally measuring the spot shape with a confocal laser microscope and calculating the amount of liquid drop from thus measured shape.

For inspecting such spot, there-can be used a visual observation (with bare eyes), a stereo microscope, a phase differential microscope, an optical microscope with a differential interference microscopic method, or an aforementioned confocal laser microscope.

Also as an item of inspection for the spotting, there can be included at least either one of whether a probe is present in a desired position, an obtaining of a present amount, and an obtaining of a probe information. The probe information in this case can be, for example, information on a base sequence in the nucleic acid of the probe, for example information on a nucleotide composition or an amino acid composition, such as an amino acid composition of a protein or an oligopeptide, and such information on the nucleic acid base sequence or the amino acid sequence in the probe can be obtained from a mass number of the probe.

More specifically, in order to investigate whether a desired probe is present in a desired position, it is necessary, in a strict sense, to investigate the nucleic acid base sequence in case of a nucleic acid probe or the amino acid sequence in case of a peptide probe, but a mass number of the probe is analyzed in the present invention, as explained in the foregoing, in case such strict investigation is difficult. For analyzing the mass number, the aforementioned MALDI method can be utilized. In such case, it is possible to execute the analysis by the MALDI method either on an actual carrier on which a probe is spotted, or on a plate for MALDI analysis on which a probe is spotted separately. It is also possible to analyze a spotted probe by the TOF-SIMS method.

In case of mass analysis of a spotted probe, there can be utilized an aforementioned method of analyzing a total mass number of the probe and identifying the probe through a comparison with a theoretical value. For such method, there can be utilized a method explained in the foregoing. More specifically, a nucleotide composition of a nucleic acid can be determined by comparing a total mass number obtained by analyzing the total mass number of nucleic acid and a total mass number calculated from a mass number, calculated in advance, of each nucleotide. Also an amino acid composition of a protein or an oligopeptide can be determined by comparing a total mass number obtained by analyzing the total mass number of protein or oligopeptide and a total mass number calculated from a mass number, calculated in advance, of each amino acid. For obtaining the total mass number of the probe, there can be utilized the MALDI method or the TOF-SIMS method. In such case, a precision of mass resolution of the total mass number of the probe and a precision of calculation of the mass number of nucleotide or amino acid are preferably equal to or higher than precisions required for specifying the nucleotide or the amino acid composition in the probe. More specifically, the precision of mass resolution of the total mass number of the probe and the precision of calculation of the mass number of nucleotide or amino acid are first place in the fraction or better, preferably second place in the fraction or better, more preferably third place in the fraction or better, and further preferably fourth place in the fraction or better.

A chip, in which the inspection on the spotting is judged “good”, is subjected to a reaction by standing for a suitable time in a condition for fixing the probe to the substrate surface, for example in case of bonding by a covalent bond, in a chamber controlled at a suitable temperature and in a saturated water vapor pressure for preventing evaporation of the liquid drop.

In the following, there will be explained an analytical inspection of the probe fixed on the substrate, constituting a final inspection item. As explained in the beginning, the probe is fixed in principle in a level of a single molecular layer on the substrate, and a matrix (spot) size may be reduced to about 10 μm, and a surface analyzing means of an extremely high sensitivity is required in such case. Also in order to investigate a spot shape etc. of the probe in the chip which has gone through the production process up to this point, a two-dimensional imaging technology of a high sensitivity is also indispensable in addition to the analysis of atoms, molecules etc.

A detection of nucleic acid in a level of a single molecular film fixed on a substrate by the TOF-SIMS method is already reported (Proceeding of the 12th International Conference on Secondary Ion Mass Spectrometry 951, 1999), and this example cites, as nucleic acid fragment ions detected by the TOF-SIMS, fragment ions formed by decomposition of bases and fragment ions formed by decomposition of a phosphoric acid backbone. In the present invention, in consideration of the aforementioned necessity, it is preferred to employ the TOF-SIMS method as one of the means for analyzing the probe fixed on the substrate. The TOF-SIMS method, being a surface analyzing method of an extremely high sensitivity as explained in the foregoing, is extremely adequate for analyzing the probe formed on the chip of the present invention. The TOF-SIMS method, being capable also of two-dimensional image, is further suitable for the present invention. More specifically, the analysis by the TOF-SIMS method can be executed while imaging the probes spotted on the carrier in spot shapes.

As explained in the foregoing, the TOF-SIMS method can utilize a function of electrically neutralizing the substrate surface thereby preventing a charge-up phenomenon at the measurement, and can thus execute measurement on a surface of a relatively low conductivity such as of glass, but the two-dimensional imaging may be more affected by the charge-up phenomenon, and, in such case, the present invention executes the analysis of the chip utilizing means for preventing charge-up by a random scanning of the primary ions (random raster scan).

The TOF-SIMS method analyzes secondary ions derived from the analyzed substance and generated by the irradiation with the primary ions as explained in the foregoing, and, in the probe chip, it is preferable to select suitable ions derived from the probe, in consideration of an ionization efficiency, a sensitivity and an S/N ratio. In the present invention, for example in a nucleic acid probe, the probe analysis including the two-dimensional imaging is executed on either one of P⁻, PO⁻, PO²⁻ and PO³⁻ or a sum of plural values as the secondary ion data. It is also possible, based on the result of two-dimensional imaging, to analyze a probe amount within a spot (arrangement density). Such analysis enables a more detailed evaluation of the produced chip.

Also specified secondary ions derived from the probe are preferably those enabling to determine the total mass number of the probe based on the mass number of such secondary ions. In such case, it is possible to further determine a nucleotide composition or an amino acid composition of the probe through a comparison of the determined total mass number of the probe and the calculated mass numbers of the nucleotides in case of a nucleic acid probe or the calculated mass numbers of the amino acids in case of an amino acid probe. Also in such case, a precision of mass resolution of the total mass number of the probe and a precision of calculation of the mass numbers of nucleotides or amino acids are preferably equal to or higher than precisions required for specifying the compositions of the nucleotide or the amino acid in the probe. More specifically, the precision of mass resolution of the total mass number of the probe and the precision of calculation of the mass number of nucleotide or amino acid are first place in the fraction or better, preferably second place in the fraction or better, more preferably third place in the fraction or better, and further preferably fourth place in the fraction or better.

As the primary ions to be used in various analyses in the TOF-SIMS method, there can be utilized ions of cerium, gallium, or gold or polyvalent gold ions.

For analyzing the probe, there can also be employed the XPS method explained in the foregoing. The XPS method, through inferior in sensitivity to the TOF-SIMS method, can provide different information. It is also capable of two-dimensional imaging, and can therefore be utilized for the probe analysis. More specifically, for analyzing the probe fixed on the carrier, there can be utilized an imaging with the XPS method. For such imaging with the XPS method, there can be utilized a method of executing a scan with a soft X-ray focused to about 10 μm to obtain a two-dimensional image of photoelectrons, or a method of irradiating an analyzed sample with a non-condensed soft X-ray and projecting emitting photoelectrons with an electron lens thereby obtaining a two-dimensional image.

The MALDI method can also be utilized as another probe analyzing method. The MALDI method, being capable of detecting secondary ions of an analyzed substance as explained before, is extremely suitable for analyzing a probe in a spot of the chip. However, in case the probe is fixed to the substrate by a covalent bond, the inspected substance may not be desorbed and ionized by a laser irradiation only, and, in such case, there is employed a method, in the present invention, of fixing the probe to the substrate by a structure which can be cleaved by the laser irradiation whereby such structure is cleaved by the laser irradiation for measurement and the probe is desorbed and ionized.

In case of employing the MALDI method, it is preferred that at least an entire probe portion of the probe is covalent bonded to the substrate by a structure that can be cleaved from the carrier surface by the laser light employed in the MALDI method. It is also preferred that a mass number obtained by the MALDI method enables to obtain the total number of the probe. With such selection of the mass number, it is possible to further determine a nucleotide composition or an amino acid composition of the probe through a comparison of the determined total mass number of the probe and the calculated mass numbers of the nucleotides in case of a nucleic acid probe or the calculated mass numbers of the amino acids in case of an amino acid probe. Also in such case, a precision of mass resolution of the total mass number of the probe and a precision of calculation of the mass numbers of nucleotides or amino acids are preferably equal to or higher than precisions required for specifying the compositions of the nucleotide or the amino acid in the probe. More specifically, the precision of mass resolution of the total mass number of the probe and the precision of calculation of the mass number of nucleotide or amino acid are first place in the fraction or better, preferably second place in the fraction or better, more preferably third place in the fraction or better, and further preferably fourth place in the fraction or better.

Also in the analysis of the probe fixed to the substrate, there can be employed the aforementioned method of identifying the probe through the comparison of the analyzed total mass number of the probe and the theoretical value.

In the present invention, these probe analyzing methods are used for a qualitative or quantitative analysis of the probe or a two-dimensional imaging of probe spots, and a judgment of “good” or “not good” state is made according to a predetermined criterion, thereby providing a quality assurance for forwarding of the chip.

In addition, in the present invention, an inspection and a quality assurance of the ship are made principally by two-dimensional imaging (partial in certain cases) with a SPM (scanning probe microscopy) method including an AFM (atomic force microscope) method, an SECM (scanning electrochemical microscopy) method or an ESEM (environmental scanning electron microscopy) method. The ESEM method, being capable of measurement in a low vacuum or in a saturated vapor pressure in such low vacuum, allows the chip after the analysis to be used.

In the foregoing, there have been explained a producing method for a probe chip of the present invention, a producing apparatus (or apparatuses) therefor, a probe chip of the present invention, and a quality assuring method of the present invention for a probe chip, but the quality assuring method for the probe chip can have various levels according to the requirement, for example a case of executing analytical inspections on all the items of a probe in a probe solution prior to spotting, a surface treated substrate, a spot after spotting and a probe fixed to the substrate after spotting, utilizing the producing method and the producing apparatus (or apparatuses) explained in the foregoing, or a case of executing analytical inspections on a part of such items. There can also be conceived a case of inspecting all such items on all the chips or a case of inspecting a part of the chips. There can also be conceived a case of inspecting all the probe spots on a chip, or a case of inspecting a part of the probe spots. In the present invention, the quality assurance of the probe chip is achieved by executing such analyses according to the situation and the necessity.

Also, all the steps or a part thereof may be automated. Further, the aforementioned apparatuses may be so arranged that a series of steps can be executed in succession as a manufacturing line, or arranged as a group of apparatuses as in so-called batch system in a semiconductor manufacturing process.

### INDUSTRIAL APPLICABILITY

The method of the present invention enables production of a probe chip with a sufficiently assured quality.

1. A method for producing a probe carrier comprising:
(1) a step of preparing a purified probe;
(2) a step of obtaining probe information on the purified probe;
(3) a step of judging “good” or “not good” quality of each purified probe according to the obtained probe information and a predetermined criterion;
(4) a step of obtaining a probe of which quality is “good” in case of the purified probe of which quality is judged as “not good”;
(5) a step of individually dissolving each purified probe judged as “good” in a solvent for ejection to a carrier, based on at least a part of the probe information obtained in (2), at a predetermined concentration and storing each obtained probe solution in an individual storing container;
(6) a step of transferring each probe solution stored in said storing container to another container equipped in an apparatus for deposition onto the carrier;
(7) a step of applying a surface treatment to the carrier for fixing the probe;
(8) a step of depositing said probe solution onto a treated surface of said carrier by a method including following steps, thereby forming a plurality of mutually independent probe fixation areas;
(8-1) a step of executing an analytical inspection on the carrier subjected to said surface treatment and judging “good” or “not good” state of said carrier according to the result of said analytical inspection and a predetermined criterion;
(8-2) a step of depositing at least one selected from said plural probe solutions onto the carrier judged as “good” so as to form a probe deposition area independent for each probe solution;
(8-3) a step of executing an inspection, concerning a formed state of the probe deposition area, on the carrier on which said probe deposition area is formed, and judging “good” or “not good” state of said deposition according the result of said inspection and a predetermined criterion;
(8-4) a step of executing, on the carrier having the probe deposition area judged as “good”, a fixation of the probe to the surface of the carrier thereby obtaining a probe carrier;
(8-5) a step of executing an analytical inspection on the probe in at least one of the plural probe fixation areas constituted of probes fixed on said carrier; and
(8-6) a step of judging “good” or “not good” state of the produced probe carrier according to the result of said analytical inspection and a predetermined criterion.

2. A method for producing a probe carrier comprising:
(a) a step of designing plural kinds of probes for detecting a target substance;
(b) a step of synthesizing the designed plural probes;
(c) a step of individually purifying the synthesized plural probes;
(d) a step of obtaining probe information on each purified probe;
(e) a step of judging “good” or “not good” state of synthesis and purification in each purified probe according to the obtained probe information and a predetermined criterion;
(f) a step of repeating the foregoing steps (b) to (e) on the purified probe of which state of synthesis and purification is judged as “not good”, thereby obtaining “good” state of synthesis and purification in all the purified probes;
(g) a step of individually dissolving each purified probe judged as “good” in a solvent for ejection to a carrier, based on at least a part of the probe information obtained in (d), in a predetermined concentration and storing each obtained probe solution in an individual storing container;
(h) a step of transferring each probe solution stored in the storing container to another container equipped in an apparatus for deposition onto the carrier;
(i) a step of applying a surface treatment for fixing the probe to the carrier;
(j) a step of depositing the probe solution onto a treated surface of the carrier by a method including following steps, thereby forming a plurality of mutually independent probe fixation areas;
(j-1) a step of executing an analytical inspection on the carrier for judging “good” or “not good” state of the carrier according to the result of the analytical inspection and a predetermined criterion;
(j-2) a step of depositing at least one selected from plural probe solutions onto the carrier judged as “good” so as to form a probe deposition area independent for each probe solution;
(j-3) a step of executing an inspection, concerning a formed state of the probe deposition area, on the carrier on which the probe deposition area is formed, and judging “good” or “not good” state of the deposition according the result of the inspection and a predetermined criterion;
(j-4) a step of executing, on the carrier having the probe deposition area judged as “good”, a fixation of the probe to the surface of the carrier thereby obtaining a probe carrier;
(j-5) a step of executing an analytical inspection on the probe in at least one of the plural probe fixation areas constituted of probes fixed on the carrier; and
(j-6) a step of judging “good” or “not good” state of the produced probe carrier according to the result of the analytical inspection and a predetermined criterion.

3. A producing method according to claim 1, wherein the probe is a nucleic acid.

4. A producing method according to claim 1, wherein the probe information is a weight of said probe.

5. A producing method according to claim 1, wherein the probe information is a purity of said probe.

6. A producing method according to claim 1, wherein the probe information is base sequence information of a nucleic acid.

7. A producing method according to claim 1, wherein a fixation of the probe to the carrier is executed by a covalent bonding.

8. A producing method according to claim 1, wherein means for spotting the probe is an apparatus equipped with single or plural ink jet nozzles.

9. A producing system to be employed in a method for producing a probe carrier according to claim 1, comprising:
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

10. A producing system to be employed in a method for producing a probe carrier according to claim 1, comprising:
a synthesis apparatus for synthesizing plural designed probes;
a purification apparatus for individually purifying the synthesized plural probes;
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

11. A quality assurance method for a probe carrier, wherein a producing method according to claim 1 is employed to execute an analytical inspection on any one or all of a probe in a probe solution prior to the deposition onto a carrier, a surface treated carrier, a probe deposition area after the deposition of a probe solution, and a probe fixed to a carrier after the deposition of a probe solution, thereby assuring a quality of a probe chip.

12. A quality assurance method according to claim 11, wherein the probe chip is a probe carrier produced by
(1) a step of preparing a purified probe;
(2) a step of obtaining probe information on the purified probe;
(3) a step of judging “good” or “not good” quality of each purified probe
(4) a step of obtaining a probe of which quality is “good” in case of the purified probe of which quality is judged as “not good”;
(5) a step of individually dissolving each purified probe judged as “good” in a solvent for ejection to a carrier, based on at least a part of the probe information obtained in (2), at a predetermined concentration and storing each obtained probe solution in an individual storing container;
(6) a step of transferring each probe solution stored in said storing container to another container equipped in an apparatus for deposition onto the carrier;
(7) a step of applying a surface treatment to the carrier for fixing the probe;
(8) a step of depositing said probe solution onto a treated surface of said carrier by a method including following steps, thereby forming a plurality of mutually independent probe fixation areas;
(8-1) a step of executing an analytical inspection on the carrier subjected to said surface treatment and judging “good” or “not good” state of said carrier according to the result of said analytical inspection and a predetermined criterion;
(8-2) a step of depositing at least one selected from said plural probe solutions onto the carrier judged as “good” so as to form a probe deposition area independent for each probe solution;
(8-3) a step of executing an inspection, concerning a formed state of the probe deposition area, on the carrier on which said probe deposition area is formed, and judging “good” or “not good” state of said deposition according the result of said inspection and a predetermined criterion;
(8-4) a step of executing, on the carrier having the probe deposition area judged as “good”, a fixation of the probe to the surface of the carrier thereby obtaining a probe carrier;
(8-5) a step of executing an analytical inspection on the probe in at least one of the plural probe fixation areas constituted of probes fixed on said carrier; and
(8-6) a step of judging “good” or “not good” state of the produced probe carrier according to the result of said analytical inspection and a predetermined criterion.

13. A probe carrier produced by a producing method according to claim 1.

14. A probe carrier of which quality is assured by a quality assurance method according to claim 11.

15. A method for producing a probe carrier having plural probes fixed on a surface of a carrier, comprising:
a step of executing an analytical inspection on a surface of said carrier and judging “good” or “not good” state of said carrier according to the result of said analytical inspection and a predetermined criterion;
a step of depositing at least one selected from plural probe solutions onto said carrier judged as “good” so as to form a probe deposition area independent for each probe solution;
a step of executing an inspection, concerning a formed state of said probe deposition area, on the carrier on which said probe deposition area is formed, and judging “good” or “not good” state of said deposition according the result of said inspection and a predetermined criterion;
a step of executing, on the carrier having said probe deposition area judged as “good”, a fixation of the probe to the surface of the carrier thereby obtaining a probe carrier;
a step of executing an analytical inspection on the probe in at least one of the plural probe fixation areas constituted of probes fixed on said carrier; and
a step of judging “good” or “not good” state of the produced probe carrier according to the result of said analytical inspection and a predetermined criterion.

16. A producing system to be employed in a method for producing a probe carrier according to claim 2, comprising:
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

17. A producing system to be employed in a method for producing a probe carrier according to claim 3, comprising:
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

18. A producing system to be employed in a method for producing a probe carrier according to claim 4, comprising:
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

19. A producing system to be employed in a method for producing a probe carrier according to claim 5, comprising:
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

20. A producing system to be employed in a method for producing a probe carrier according to claim 6, comprising:
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

21. A producing system to be employed in a method for producing a probe carrier according to claim 7, comprising:
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

22. A producing system to be employed in a method for producing a probe carrier according to claim 8, comprising:
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

23. A producing system to be employed in a method for producing a probe carrier according to claim 2, comprising:
a synthesis apparatus for synthesizing plural designed probes;
a purification apparatus for individually purifying the synthesized plural probes;
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

24. A producing system to be employed in a method for producing a probe carrier according to claim 3, comprising:
a synthesis apparatus for synthesizing plural designed probes;
a purification apparatus for individually purifying the synthesized plural probes;
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

25. A producing system to be employed in a method for producing a probe carrier according to claim 4, comprising:
a synthesis apparatus for synthesizing plural designed probes;
a purification apparatus for individually purifying the synthesized plural probes;
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

26. A producing system to be employed in a method for producing a probe carrier according to claim 5, comprising:
a synthesis apparatus for synthesizing plural designed probes;
a purification apparatus for individually purifying the synthesized plural probes;
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

27. A producing system to be employed in a method for producing a probe carrier according to claim 6, comprising:
a synthesis apparatus for synthesizing plural designed probes;
a purification apparatus for individually purifying the synthesized plural probes;
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

28. A producing system to be employed in a method for producing a probe carrier according to claim 7, comprising:
a synthesis apparatus for synthesizing plural designed probes;
a purification apparatus for individually purifying the synthesized plural probes;
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

29. A producing system to be employed in a method for producing a probe carrier according to claim 8, comprising:
a synthesis apparatus for synthesizing plural designed probes;
a purification apparatus for individually purifying the synthesized plural probes;
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

30. A quality assurance method for a probe carrier, wherein a producing method according to claim 2 is employed to execute an analytical inspection on any one or all of a probe in a probe solution prior to the deposition onto a carrier, a surface treated carrier, a probe deposition area after the deposition of a probe solution, and a probe fixed to a carrier after the deposition of a probe solution, thereby assuring a quality of a probe chip.

31. A quality assurance method for a probe carrier, wherein a producing method according to claim 3 is employed to execute an analytical inspection on any one or all of a probe in a probe solution prior to the deposition onto a carrier, a surface treated carrier, a probe deposition area after the deposition of a probe solution, and a probe fixed to a carrier after the deposition of a probe solution, thereby assuring a quality of a probe chip.

32. A quality assurance method for a probe carrier, wherein a producing method according to claim 4 is employed to execute an analytical inspection on any one or all of a probe in a probe solution prior to the deposition onto a carrier, a surface treated carrier, a probe deposition area after the deposition of a probe solution, and a probe fixed to a carrier after the deposition of a probe solution, thereby assuring a quality of a probe chip.

33. A quality assurance method for a probe carrier, wherein a producing method according to claim 5 is employed to execute an analytical inspection on any one or all of a probe in a probe solution prior to the deposition onto a carrier, a surface treated carrier, a probe deposition area after the deposition of a probe solution, and a probe fixed to a carrier after the deposition of a probe solution, thereby assuring a quality of a probe chip.

34. A quality assurance method for a probe carrier, wherein a producing method according claim 6 is employed to execute an analytical inspection on any one or all of a probe in a probe solution prior to the deposition onto a carrier, a surface treated carrier, a probe deposition area after the deposition of a probe solution, and a probe fixed to a carrier after the deposition of a probe solution, thereby assuring a quality of a probe chip.

35. A quality assurance method for a probe carrier, wherein a producing method according to claim 7 is employed to execute an analytical inspection on any one or all of a probe in a probe solution prior to the deposition onto a carrier, a surface treated carrier, a probe deposition area after the deposition of a probe solution, and a probe fixed to a carrier after the deposition of a probe solution, thereby assuring a quality of a probe chip.

36. A quality assurance method for a probe carrier, wherein a producing method according to claim 8 is employed to execute an analytical inspection on any one or all of a probe in a probe solution prior to the deposition onto a carrier, a surface treated carrier, a probe deposition area after the deposition of a probe solution, and a probe fixed to a carrier after the deposition of a probe solution, thereby assuring a quality of a probe chip.

37. A quality assurance method for a probe carrier, wherein a producing system according to claim 9 is employed to execute an analytical inspection on any one or all of a probe in a probe solution prior to the deposition onto a carrier, a surface treated carrier, a probe deposition area after the deposition of a probe solution, and a probe fixed to a carrier after the deposition of a probe solution, thereby assuring a quality of a probe chip.

38. A quality assurance method for a probe carrier, wherein a producing system according to claim 10 is employed to execute an analytical inspection on any one or all of a probe in a probe solution prior to the deposition onto a carrier, a surface treated carrier, a probe deposition area after the deposition of a probe solution, and a probe fixed to a carrier after the deposition of a probe solution, thereby assuring a quality of a probe chip.

39. A quality assurance method according to claim 1, wherein the probe chip is a probe carrier produced by a
(a) a step of designing plural kinds of probes for detecting a target substance;
(b) a step of synthesizing the designed plural probes;
(c) a step of individually purifying the synthesized plural probes;
(d) a step of obtaining probe information on each purified probe;
(e) a step of judging “good” or “not good” state of synthesis and purification in each purified probe according to the obtained probe information and a predetermined criterion;
(f) a step of repeating the foregoing steps (b) to (e) on the purified probe of which state of synthesis and purification is judged as “not good”, thereby obtaining “good” state of synthesis and purification in all the purified probes;
(g) a step of individually dissolving each purified probe judged as “good” in a solvent for ejection to a carrier, based on at least a part of the probe information obtained in (d), in a predetermined concentration and storing each obtained probe solution in an individual storing container;
(h) a step of transferring each probe solution stored in the storing container to another container equipped in an apparatus for deposition onto the carrier;
(i) a step of applying a surface treatment for fixing the probe to the carrier;
(j) a step of depositing the probe solution onto a treated surface of the carrier by a method including following steps, thereby forming a plurality of mutually independent probe fixation areas;
(j-1) a step of executing an analytical inspection on the carrier for judging “good” or “not good” state of the carrier according to the result of the analytical inspection and a predetermined criterion;
(j-2) a step of depositing at least one selected from plural probe solutions onto the carrier judged as “good” so as to form a probe deposition area independent for each probe solution;
(j-3) a step of executing an inspection, concerning a formed state of the probe deposition area, on the carrier on which the probe deposition area is formed, and judging “good” or “not good” state of the deposition according the result of the inspection and a predetermined criterion;
(j-4) a step of executing, on the carrier having the probe deposition area judged as “good”, a fixation of the probe to the surface of the carrier thereby obtaining a probe carrier;
(j-5) a step of executing an analytical inspection on the probe in at least one of the plural probe fixation areas constituted of probes fixed on the carrier; and
(j-6) a step of judging “good” or “not good” state of the produced probe carrier according to the result of the analytical inspection and a predetermined criterion.

40. A quality assurance method according to claim 12, wherein the probe chip is a probe carrier wherein the probe is a nucleic acid.

41. A quality assurance method according to claim 12, wherein the probe chip is a probe carrier wherein the probe information is a weight of said probe.

42. A quality assurance method according to claim 12, wherein the probe chips a probe carrier produced wherein the probe information is purity of said probe.

43. A quality assurance method according to claim 12, wherein the probe chip is a probe carrier wherein the probe information is base sequence information of a nucleic acid.

44. A quality assurance method according to claim 12, wherein the probe chip is a probe carrier wherein a fixation of the probe to the carrier is executed by a covalent bonding.

45. A quality assurance method according to claim 12, wherein the probe chip is a probe carrier wherein means for spotting the probe is an apparatus equipped with single or plural ink jet nozzles.

46. A quality assurance method according to claim 11, wherein the probe chip is a probe carrier comprising:
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

47. A quality assurance method according to claim 11, wherein the probe chip is a probe carrier produced by a producing system comprising:
a synthesis apparatus for synthesizing plural designed probes;
a purification apparatus for individually purifying the synthesized plural probes;
an analysis apparatus for obtaining the probe information of each purified probe;
an inspection apparatus for judging “good” or “not good” state of synthesis and purification of each purified probe;
an apparatus for depositing, to the carrier, each probe solution supplied from a storing container individually storing each purified probe solution judged as “good”;
an analysis apparatus for the carrier subjected to said surface treatment;
an inspection apparatus for judging “good” or “not good” state, concerning a formed state of the probe deposition area, on the carrier on which the probe deposition area is formed;
an apparatus for executing, on the carrier having the probe deposition area judged as “good”, a fixation of the probe to the surface of the carrier thereby obtaining a probe carrier; and
an apparatus for executing an analytical inspection on the probe in at least one of the plural probe fixation areas constituted of probes fixed on the carrier.

48. A probe carrier produced by a producing method according to claim 2.

49. A probe carrier produced by a producing method according to claim 3.

50. A probe carrier produced by a producing method according to claim 4.

51. A probe carrier produced by a producing method according to claim 5.

52. A probe carrier produced by a producing method according to claim 6.

53. A probe carrier produced by a producing method according to claim 7.

54. A probe carrier produced by a producing method according to claim 8.

55. A probe carrier produced by a producing system according to claim 9.

56. A probe carrier produced by a producing system according to claim 10.
